(19) United States
(12) Patent Application Publication
(10) Pub. No.: US 2017/0340296 A1
(43) Pub. Date: Nov. 30, 2017

(54) SMART APPARATUS FOR ACQUIRING PATIENT IMAGES

(71) Applicant: HDX Corporation, Seoul (KR)

(72) Inventors: Dong Oh SHIN, Seoul (KR); Hyun Do HUH, Bucheon-si, Gyeonggi-do (KR)

(73) Assignee: HDX Corporation, Seoul (KR)

(21) Appl. No.: 15/532,784

(22) PCT Filed: May 18, 2015
(86) PCT No.: PCT/KR2015/004977

§ 371 Date: Jun. 2, 2017

(30) Foreign Application Priority Data
Dec. 2, 2014 (KR) 10-2014-0170719

(51) Int. Cl. A61B 6/03; A61B 6/04; A61B 6/02

(52) U.S. Cl. CPC A61B 6/035; A61B 6/027; A61B 6/0407

(57) ABSTRACT

The present invention relates to a smart apparatus for acquiring patient images, the smart apparatus being structured so as to integrate capabilities for acquiring two-dimensional images and three-dimensional images into a single piece of equipment, thereby allowing the expense and installation space therefor to be minimized. The smart apparatus for acquiring patient images according to the present invention comprises: a gantry having a cylindrical opening; CT X-ray tube and curved X-ray detector installed in the gantry 180 degrees apart and installed so as to be rotatable along the circumferential direction of the gantry to acquire three-dimensional images of a person being treated accommodated in the interior of the opening of the gantry by rotating around the person; two-dimensional X-ray tube and X-ray detector installed in the gantry 180 degrees apart and installed, along with the CT X-ray tube and curved X-ray detector, so as to be rotatable along the circumferential direction of the gantry to acquire two-dimensional x-ray images of a person being treated accommodated in the interior of the opening of the gantry; a rotation means for simultaneously rotating the CT X-ray tube and X-ray detector and two-dimensional X-ray tube and X-ray detector along the circumferential direction of the gantry; a couch disposed on one side of the gantry so as to be horizontally movable in and out of the opening of the gantry and on which the person to be treated is placed.

### TECHNICAL FIELD

The present invention relates to an apparatus capable of acquiring an image that is optimized for simulation planning of cancer treatment or the like as well as simultaneously acquiring a 2-dimensional (2D) image and a 3D image during general treatment for a patient, and more particularly, to a smart apparatus for acquiring patient images that is capable of acquiring an image in an optimized range through a 2D image of a treated person and utilizing the acquired image in diagnosis and radiation treatment planning, easily moving and checking a radiation treatment site before radiation treatment, and acquiring a patient's image information such as a general 2D image and a 3D or 4D (respiration-gated) treatment planning image by a single apparatus.

### BACKGROUND ART

Generally, radiation treatment includes computerized tomography (CT) scanning using a CT simulator, treatment planning based on a CT image, imaging using a 2-dimensional (2D) X-ray simulator, treatment planning based on a 2D image, and actual radiation treatment.

A CT simulator is an apparatus that produces cross-sectional images of an inside of the human body by using a rotating x-ray tube and a detector. A CT image scanned by the CT simulator may record even small differences in soft tissues (blood, cerebrospinal fluid, gray matter, white matter, tumors, etc.) that cannot be seen from a general X-ray image, and obtained data may be reconstructed to form a 3D image.

Generally, in CT scanning, unlike in general X-ray imaging, a person lies down in a large machine with a cylindrical hole, and an X-ray generating apparatus performs imaging by rotating in a circle around the person from outside of the cylindrical hole. While various front and rear images overlap each other in general X-ray imaging, when CT scanning is performed, imaging equipment uses a computer to measure X-rays transmitted at various angles to the human body by an imaging apparatus, that shows cross-sectional images of a certain part of the body, and reconstructs absorption values related to cross-sections of the human body and shows the reconstructed absorption values as an image.

However, conventionally, because a CT simulator and a 2D X-ray simulator are manufactured as separate pieces of equipment and used, there are problems such as an increase in cost and an increase in an installation space due to separately purchasing the CT simulator and the 2D X-ray simulator and an inconvenience of having to move a patient.

To solve the above problems, in Japanese Unexamined Patent Application, First Publication No. H10-179565, a treatment planning apparatus in which a CT simulator and a 2D X-ray simulator are adjacently arranged at a 90-degree-interval, and a placing table on which a patient is placed is configured to rotate about an axis perpendicular thereto so that simulation and treatment plans can be established while the patient is moved to a position corresponding to the CT simulator and a position corresponding to the 2D x-ray simulator is disclosed.

However, in the above-described treatment planning apparatus, because a CT simulator and a 2D X-ray simulator still need to be separately manufactured, there is a problem in that the problems of an increase in cost and an increase in an installation space still exist.

Also, because an X-ray tube for image acquisition does not rotate during 2D X-ray image acquisition that is generally performed in the radiology field, there is an inconvenience of having to move a patient to acquire an image.

### DISCLOSURE

### Technical Problem

The present invention is for solving the above problems, and an objective of the present invention is to provide a smart apparatus for acquiring patient images in which a function of acquiring a 2-dimensional (2D) image of a patient and a function of acquiring a 3D or 4D (respiration-gated) computerized tomography (CT) image can be implemented by a single apparatus so that cost and an installation space can be minimized.

### Technical Solution

To solve the above objective, according to the present invention, a smart apparatus for acquiring patient images includes a gantry having a cylindrical opening formed therein; a computerized tomography (CT) X-ray tube and a curved CT X-ray detector installed in the gantry to be spaced apart at a 180-degree-interval and rotatably installed in a circumferential direction of the gantry to acquire a 3-dimensional (3D) image while rotating around a treated person accommodated inside the opening of the gantry; a 2D X-ray tube and a 2D X-ray detector installed in the gantry to be spaced apart at a 180-degree interval and rotatably installed in the circumferential direction of the gantry together with the CT X-ray tube and the CT X-ray detector to acquire a 2D fluoroscopic image of the treated person accommodated inside the opening of the gantry; a rotator configured to simultaneously rotate the CT X-ray tube, the CT X-ray detector, the 2D X-ray tube, and the 2D X-ray detector in the circumferential direction of the gantry; and a couch installed at one side of the gantry to be horizontally movable inside and outside the opening and on which the treated person is placed.

### Advantageous Effects

According to the present invention, in a smart apparatus for acquiring patient images, a computerized tomography (CT) X-ray tube and a CT X-ray detector configured to perform a function of acquiring a 3-dimensional (3D) or 4D CT image and a 2D X-ray tube and a 2D X-ray detector configured to acquire a 2D image are integrally configured in a gantry to rotate together, and a 2D-image acquisition function and a 3D (or 4D) CT image acquisition function can be selectively performed.

Therefore, costs of manufacturing, purchasing, and operating image acquisition equipment can be lowered, and an installation space and a shielding facility for the equipment can be reduced.

Because two image acquisition functions can be performed by a single piece of equipment without a patient being moved to pieces of equipment, and a 2D X-ray tube and a detector can acquire a 2D image while rotating, convenience of a treated person and a treating person can be improved.

### DESCRIPTION OF DRAWINGS

FIG. 1 is a view illustrating a smart apparatus for acquiring patient images according to an embodiment of the present invention.

FIG. 2 is a front view of the smart apparatus for acquiring patient images illustrated in FIG. 1.

FIG. 3 is a longitudinal cross-sectional view of the smart apparatus for acquiring patient images illustrated in FIG. 1.

FIG. 4 is a transverse cross-sectional view of the smart apparatus for acquiring patient images illustrated in FIG. 1.

FIG. 5 is a front view illustrating a smart apparatus for acquiring patient images according to another embodiment of the present invention.

### MODES OF THE INVENTION

Hereinafter, exemplary embodiments of a smart apparatus for acquiring patient images according to the present invention will be described in detail with reference to the accompanying drawings.

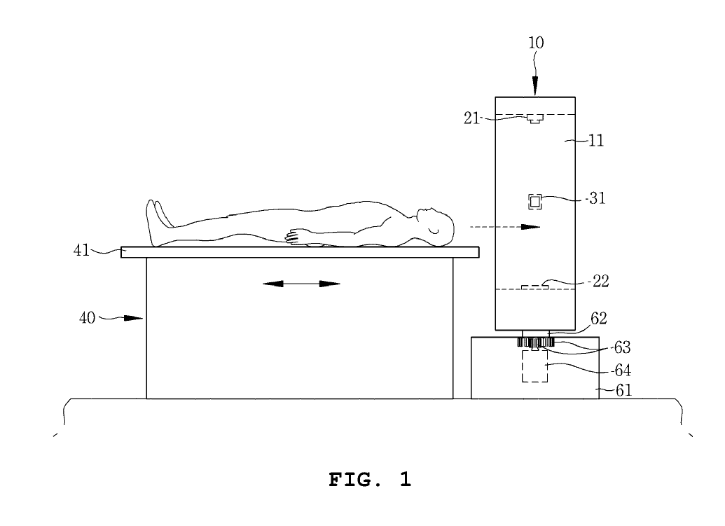

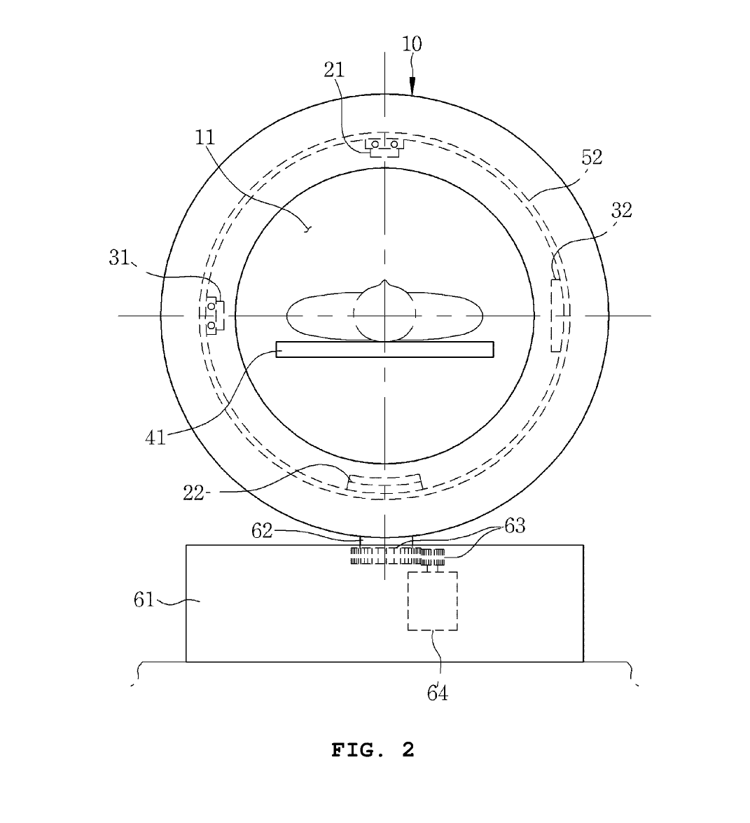

FIGS. 1 to 4 are views illustrating the smart apparatus for acquiring patient images according to an embodiment of the present invention, and the smart apparatus for acquiring patient images according to the embodiment includes a gantry 10 having a cylindrical opening 11 formed therein, a computerized tomography (CT) X-ray tube 21 and a curved CT X-ray detector 22 installed in the gantry 10 to face each other, a 2D X-ray tube 31 and a 2D X-ray detector 32 installed between the CT X-ray tube 21 and the CT X-ray detector 22 to face each other, a rotator configured to simultaneously rotate the CT X-ray tube 21, the CT X-ray detector 22, the 2D X-ray tube 31, and the 2D X-ray detector 32 in the circumferential direction of the gantry 10, a couch 40 installed at one side of the gantry 10 to horizontally move inside and outside the opening 11 and on which a treated person is placed, and a yawing rotator configured to rotate the gantry 10 back and forth in two directions about an axis perpendicular to the ground within a predetermined angle range.

The couch 40 is configured so that a bed 41 on which the treated person is placed slides in a direction horizontal to the ground, and because a known couch 40 that is installed in a conventional CT simulation apparatus can be employed as the couch 40, description of the detailed configuration and operation thereof will be omitted.

The CT X-ray tube 21 and the CT X-ray detector 22 are installed in the gantry 10 to be spaced apart at a 180-degree-interval and face each other, and rotate in the circumferential direction of the gantry 10 by the rotator. The CT X-ray detector 22 is formed of a curved surface.

The CT X-ray tube 21 and the CT X-ray detector 22 are configured to acquire a 3D CT image while rotating around the treated person accommodated inside the opening 11 of the gantry 10, and may precisely aim at planar and stereoscopic image cross-sections, digital reconstructed radiography (DRR), and a stereoscopic contour, size, shape, etc. of a body surface, a tumor, and a major organ. Also, the CT X-ray tube 21 and the CT X-ray detector 22 may be configured to acquire a 4D image for respiration-gated radiation treatment by being operated with ANZAI System, which is a respiration-gated system, as needed.

The 2D X-ray tube 31 and the 2D X-ray detector 32 are installed in the gantry 10 to be spaced apart at a 180-degree-interval and face each other and are disposed at positions spaced apart at predetermined angles from the CT X-ray tube 21 and the CT X-ray detector 22. Although the 2D X-ray tube 31 and the 2D X-ray detector 32 are arranged at a 90-degree-interval from the CT X-ray tube 21 and the CT X-ray detector 22 in the embodiment, the arrangement interval is not limited thereto.

The 2D X-ray tube 31 and the 2D X-ray detector 32 are rotated in the circumferential direction of the gantry 10 together with the CT X-ray tube 21 and the CT X-ray detector 22 by the rotator and are disposed at set imaging positions, i.e., the top point of the gantry 10, when 2D X-ray imaging is performed. The 2D X-ray tube 31 and the 2D X-ray detector 32 perform imaging of the treated person accommodated inside the opening 11 of the gantry 10 to acquire a 2D fluoroscopic image while being fixed to the gantry 10. The 2D image acquired by the 2D X-ray tube 31 and the 2D X-ray detector 32 allows precise and safe radiation treatment by checking a radiation treatment plan and treatment site coordinates when radiation treatment is performed.

A distance D2 from a central point of the gantry 10 to the 2D X-ray tube 31 and the 2D X-ray detector 32 is set to be larger than a distance D1 from the central point of the gantry 10 to the CT X-ray tube 21 and the CT X-ray detector 22. For example, the distance D1 from the central point of the gantry 10 to the CT X-ray tube 21 and the CT X-ray detector 22 is set as 85 cm, and the distance D2 from the central point of the gantry 10 to the 2D X-ray tube 31 and the 2D X-ray detector 32 is set as 100 cm.

The rotator is configured to simultaneously rotate the CT X-ray tube 21, the CT X-ray detector 22, the 2D X-ray tube 31, and the 2D X-ray detector 32 in the circumferential direction of the gantry 10. In the embodiment, the rotator includes a ring-shaped guide rail 51 formed to extend in the circumferential direction in the gantry 10, a driving plate 52 installed to rotate along the guide rail 51 and on which the CT X-ray tube 21, the CT X-ray detector 22, the 2D X-ray tube 31, and the 2D X-ray detector 32 are installed, a ring-shaped driven gear 53 coupled to the driving plate 52, a driving gear 54 installed at one side of the gantry 10 to be engaged with the driven gear 53, and a driving motor 55 configured to rotate the driving gear 54.

Therefore, when a control signal is applied to the driving motor 55 and the driving motor 55 is operated, the driving gear 54 receives power of the driving motor 55 and rotates, and the driven gear 53 engaged with the driving gear 54 and the driving plate 52 receive rotary power and rotate along the guide rail 51. The CT X-ray tube 21, the CT X-ray detector 22, the 2D X-ray tube 31, and the 2D X-ray detector 32 installed on the driving plate 52 simultaneously rotate due to the rotation of the driving plate 52. Preferably, a roller 56 or a bearing configured to roll along the guide rail 51 is installed at one end of the driving plate 52 so that the driving plate 52 smoothly rotates along the guide rail 51.

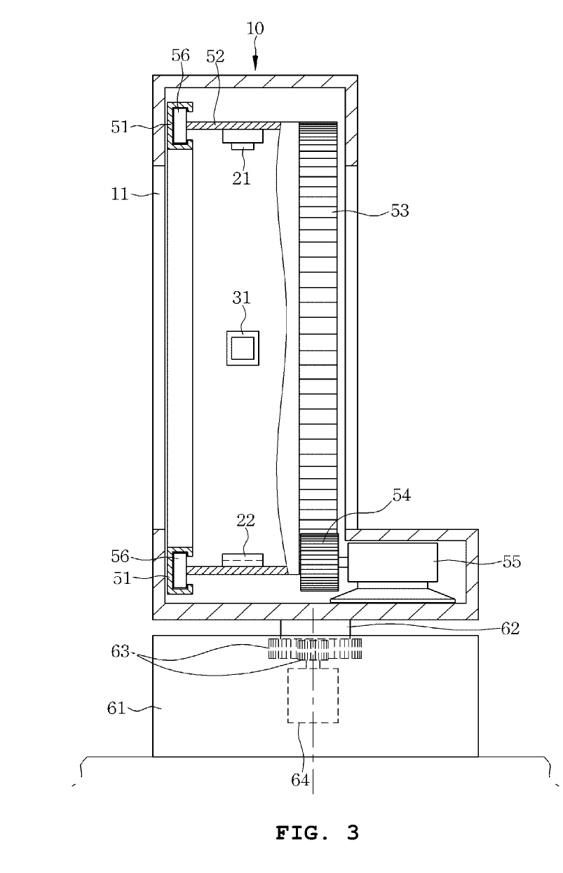

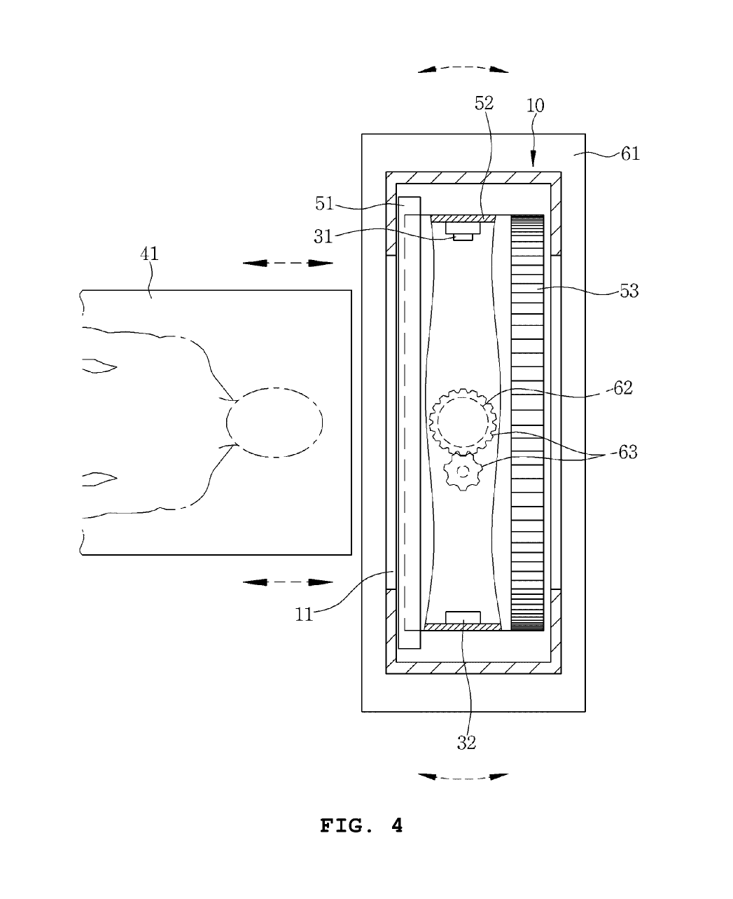

As described above, the smart apparatus for acquiring patient images according to the present invention is configured so that the gantry 10 is capable of yawing in two direction about an axis perpendicular to the ground within a predetermined angle range. As illustrated in FIGS. 3 and 4, a yawing rotator configured to generate the yawing motion of the gantry 10 may include a base 61, a rotation shaft part 62 extending vertical to the base 61, installed to rotate with respect to the base 61, and having an upper end connected to a lower end of the gantry 10, a power transmission member 63 coupled to the rotation shaft part 62, and a yaw motion motor 64 configured to transmit rotary power to the rotation shaft part 62 through the power transmission member 63.

Although a gear such as a spur gear, a bevel gear, a helical gear and a worm gear can be used as the power transmission member 63, other known power transmission mechanisms such as a timing belt or a chain may also be used.

When configured as above so that the gantry 10 is capable of yawing at a predetermined angle about an axis perpendicular to the ground, because an image can be acquired by varying a position of the treated person relative to the CT X-ray tube 21 and the CT X-ray detector 22 or the 2D X-ray tube 31 and the 2D X-ray detector 32 as needed, various precise treatment plans can be established.

Preferably, a controller configured to control operations of the CT X-ray tube 21 and the CT X-ray detector 22, the 2D X-ray tube 31 and the 2D X-ray detector 32, the rotator, and the yawing rotator is integrally configured in a single console.

The smart apparatus for acquiring patient images configured as above is operated as below.

The bed 41 of the couch 40 is horizontally moved while the treated person is placed on the couch 40 so that the treated person is accommodated inside the opening 11 of the gantry 10. The bed 41 of the couch 40 horizontally moves inside the opening 11 at a predetermined speed when CT scanning begins. Here, the driving motor 55 of the rotator is operated, and the CT X-ray tube 21 and the CT X-ray detector 22 performs imaging while rotating back and forth at a predetermined speed in the circumferential direction of the gantry 10 within a predetermined angle range (e.g.,) 185° to acquire a 3D CT image of the treated person.

Although the 2D X-ray tube 31 and the 2D X-ray detector 32 also rotate together while the CT X-ray tube 21 and the CT X-ray detector 22 rotate, X-rays are not emitted from the 2D X-ray tube 31.

When the CT image by the CT X-ray tube 21 and the CT X-ray detector 22 is acquired as above, the bed 41 of the couch 40 is horizontally moved back to the set position. Also, the driving motor 55 of the rotator is operated to move the 2D X-ray tube 31 and the 2D X-ray detector 32 to set imaging positions in the gantry 10.

When the 2D X-ray tube 31 and the 2D X-ray detector 32 are aligned at the set positions, a 2D stereoscopic image of the treated person is acquired by emitting X-rays from the 2D X-ray tube 31, and a general treatment plan or radiation treatment plan for the treated person is established.

The 3D (or 4D) CT image acquisition process by the CT X-ray tube 21 and the CT X-ray detector 22 and the 2D image acquisition process by the 2D X-ray tube 31 and the 2D X-ray detector 32 can be performed in reverse order. That is, the 2D-image acquisition by the 2D X-ray tube 31 and the 2D X-ray detector 32 can be performed first, and the 3D (or 4D) CT image acquisition by the CT X-ray tube 21 and the CT X-ray detector 22 can be performed next. Alternatively, any one of the 2D-image acquisition by the 2D X-ray tube 31 and the 2D X-ray detector 32 and the 3D (or 4D) CT image acquisition by the CT X-ray tube 21 and the CT X-ray detector 22 can be selectively performed.

In the above-described embodiment, the CT X-ray tube 21 and the CT X-ray detector 22, and the 2D X-ray tube 31 and the 2D X-ray detector 32 are installed on the single driving plate 52 to be spaced apart from the central point of the gantry 10 at different distances.

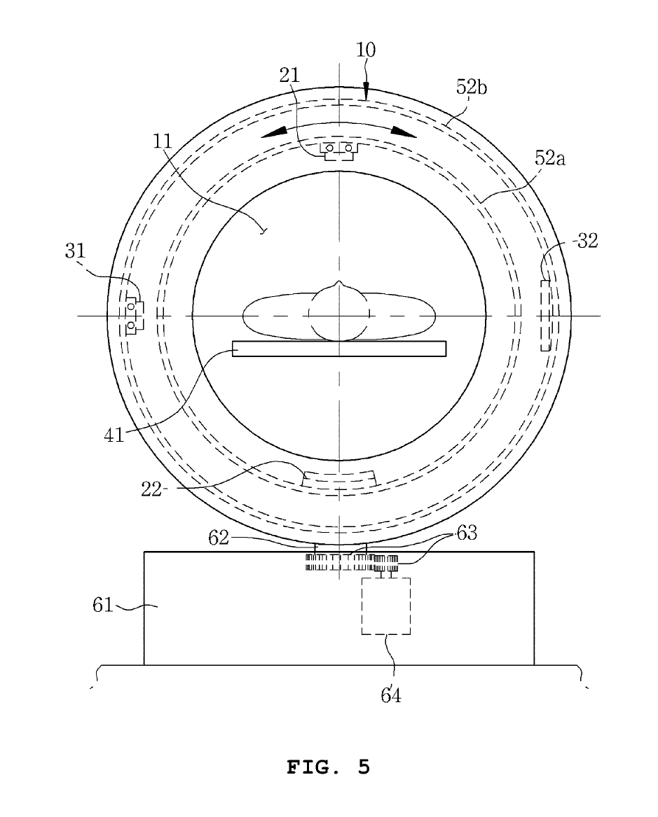

However, according to another embodiment illustrated in FIG. 5, two first and second driving plates 52a and 52b having different diameters may be rotatably installed in the gantry 10, the CT X-ray tube 21 and the CT X-ray detector 22 may be installed on the first driving plate 52a having a smaller diameter, and the 2D X-ray tube 31 and the 2D X-ray detector 32 may be installed on the second driving plate 52b having a larger diameter so that the X-ray tubes and the X-ray detectors are spaced apart from the central point of the gantry 10 at different distances.

Here, although the first and second driving plates 52a and 52b may be integrated with each other and configured to rotate together by the single driving motor 55 (see FIG. 3), the driven gear 53 (see FIG. 3), the driving gear 54 (see FIG. 3), and the driving motor 55 (see FIG. 3) may be separately installed in each of the first driving plate 52a and the second driving plate 52b, and the first driving plate 52a and the second driving plate 52b may rotate independently from each other.

In this way, in the smart apparatus for acquiring patient images according to the present invention, because the CT X-ray tube 21 and the CT X-ray detector 22 configured to perform a function of a CT simulator and the 2D X-ray tube 31 and the 2D X-ray detector 32 configured to perform a function of a 2D X-ray simulator are integrated in the gantry 10 and configured to rotate together, the CT simulation function and the 2D X-ray simulation function can be selectively performed.

Therefore, there are advantages in that costs for purchasing simulation equipment can be lowered, and an equipment installation space can be considerably reduced. Also, because two simulations can be performed by a single piece of equipment without a patient being move to pieces of equipment, convenience of a treated person and a treating person can be improved.

Although the present invention has been described in detail above with reference to embodiments, one of ordinary skill in the art to which the present invention pertains should be able to make various substitutions, additions, and modifications within the scope not departing from the above-described technical spirit, and such modified embodiments should also be understood as belong to the scope of the present invention that is defined by the claims below.

### INDUSTRIAL APPLICABILITY

The present invention is applicable to medical equipment for acquiring a patient's image information.

What is claimed is:

1. A smart apparatus for acquiring patient images, the smart apparatus comprising:
a gantry (10) having a cylindrical opening (11) formed therein;
a computerized tomography (CT) X-ray tube (21) and a curved CT X-ray detector (22) installed in the gantry (10) to be spaced apart at a 180-degree-interval and rotatably installed in a circumferential direction of the gantry (10) to acquire a 3-dimensional (3D) image while rotating around a treated person accommodated inside the opening (11) of the gantry (10);
a 2-dimensional (2D) X-ray tube (31) and a 2D X-ray detector (32) installed in the gantry (10) to be spaced apart at a 180-degree interval and rotatably installed in the circumferential direction of the gantry (10) together with the CT X-ray tube (21) and the CT X-ray detector (22) to acquire a 2D fluoroscopic image of the treated person accommodated inside the opening (11) of the gantry (10);
a rotator configured to simultaneously rotate the CT X-ray tube (21), the CT X-ray detector (22), the 2D X-ray tube (31), and the 2D X-ray detector (32) in the circumferential direction of the gantry (10); and
a couch (40) installed at one side of the gantry (10) to be horizontally movable inside and outside the opening (11) and on which the treated person is placed.

2. The smart apparatus of claim 1, further comprising a yawing rotator configured to rotate the gantry (10) back and forth in two directions about an axis perpendicular to the ground within a predetermined angle range.

3. The smart apparatus of claim 2, wherein the yawing rotator includes a base (61), a rotation shaft part (62) extending vertical to the base (61), installed to rotate with respect to the base (61), and having an upper end connected to a lower end of the gantry (10), a power transmission member (63) coupled to the rotation shaft part (62), and a yaw motion motor (64) configured to transmit rotary power to the rotation shaft part (62) through the power transmission member (63).

4. The smart apparatus of claim 1, wherein the rotator includes a ring-shaped guide rail (51) formed to extend in the circumferential direction in the gantry (10), a driving plate (52) installed to rotate along the guide rail (51) and on which the CT X-ray tube (21), the CT X-ray detector (22), the 2D X-ray tube (31), and the 2D X-ray detector (32) are installed, a ring-shaped driven gear (53) coupled to the driving plate (52), a driving gear (54) installed at one side of the gantry (10) to be engaged with the driven gear (53), and a driving motor (55) configured to rotate the driving gear (54).

5. The smart apparatus of claim 4, wherein a roller (56) configured to roll along the guide rail (51) is installed at one end of the driving plate (52).

6. The smart apparatus of claim 1, wherein a distance (D2) from a central point of the gantry (10) to the 2D X-ray tube (31) and the 2D X-ray detector (32) is set to be larger than a distance (D1) from the central point of the gantry (10) to the CT X-ray tube (21) and the CT X-ray detector (22).

7. The smart apparatus of claim 4, wherein the driving plate includes a first driving plate (52a) on which the CT X-ray tube (21) and the CT X-ray detector (22) are installed, and a second driving plate (52b) having a larger diameter than the first driving plate (52a) and on which the 2D X-ray tube (31) and the 2D X-ray detector (32) are installed.

8. The smart apparatus of claim 7, wherein the first driving plate (52a) and the second driving plate (52b) are coupled to each other and rotate together by the single driving motor (55).

9. The smart apparatus of claim 7, wherein the first driving plate (52a) and the second driving plate (52b) are configured to rotate independently from each other, and the first driving plate (52a) and the second driving plate (52b) independently rotate by receiving power from two separate driving motors (55).
